(19) United States
(12) Patent Application Publication
(10) Pub. No.: US 2016/0113506 A1
(43) Pub. Date: Apr. 28, 2016

(54) ACOUSTIC WAVE DETECTION DEVICE AND ACOUSTIC WAVE DETECTION METHOD

(71) Applicant: CANON KABUSHIKI KAISHA, Tokyo (JP)

(72) Inventors: Toru Imai, St. Louis, MO (US); Takao Nakajima, Kawasaki-shi (JP)

(21) Appl. No.: 14/884,945

(22) Filed: Oct. 16, 2015

(30) Foreign Application Priority Data
Oct. 22, 2014 (JP) 2014-215449

(51) Int. Cl. A61B 5/00

(52) U.S. Cl. CPC A61B 5/0095

(57) ABSTRACT

An acoustic wave detection device comprises an interference film formed of two opposing reflective layers; a first light source configured to irradiate light to the interference film and capable of changing a wavelength of the light; a photodetector configured to detect an intensity of reflected light; a signal acquisition unit configured to acquire an electrical signal based on a change of the intensity of the reflected light; a wavelength acquisition unit configured to acquire a reference wavelength, which is a wavelength of the measurement light emitted when measurement is performed on a first point on the interference film; and a wavelength control unit configured to determine the wavelength of the measurement light emitted when the measurement is performed on a second point on the interference film based on the reference wavelength.

### BACKGROUND OF THE INVENTION

1. Field of the Invention

The present invention relates to a device that measures the intensity of an acoustic wave.

2. Description of the Related Art

Recently, in the field of medicine, a study for imaging morphologic information or physiological information, i.e., functional information on an internal portion of an object is promoted. As one of such techniques, photoacoustic tomography (PAT) is proposed recently.

When light such as pulsed laser light is irradiated to a biological body as an object, an acoustic wave (typically, an ultrasonic wave) is generated when the light is absorbed in a biological tissue in the object. This phenomenon is called a photoacoustic effect, and the acoustic wave generated by the photoacoustic effect is called a photoacoustic wave. The tissues constituting the object have different absorptivities of light energy, and hence the sound pressure of the generated photoacoustic wave differs. In the PAT, the generated photoacoustic wave is received by an acoustic wave detector and a received signal is mathematically analyzed, whereby it is possible to image optical characteristics of the internal portion of the object, particularly a light energy absorption density distribution.

In the photoacoustic tomography, since acoustic waves having different frequencies are generated depending on an object that absorbs the light, it is necessary to use the acoustic wave detector having a wide band.

As such an acoustic wave detector, a transducer that uses a piezoelectric phenomenon or change of a capacity is used and, in recent years, the acoustic wave detector that uses resonance of light is developed. For example, when the acoustic wave is made incident on an optical interference film in a state in which light (hereinafter referred to as measurement light) is irradiated to the optical interference film, the intensity of reflected light changes as a sound pressure changes. Accordingly, by detecting the intensity of the reflected light using a light detecting sensor such as a photodiode, it is possible to detect the change of the sound pressure of the acoustic wave. A Fabry-Perot interference film is the representative optical interference film.

As the invention relevant to this, Non patent Literature 1, for example, describes a device that moves an irradiation position of the measurement light on the Fabry-Perot interference film and detects the acoustic wave at a plurality of positions on the interference film.

Such an acoustic wave detector that uses the resonance of light is capable of detecting the acoustic wave in a wide band, and hence it can be said that the acoustic wave detector is suitable for the photoacoustic tomography.

Patent Literature 1: Japanese Patent Application Laid-open No. 2004-003969

Non Patent Literature 1: Edward Zhang, Jan Laufer, and Paul Beard,“Backward-mode multiwavelength photoacoustic scanner using a planar Fabry-Perot polymer film ultrasound sensor for high-resolution three-dimensional imaging of biological tissues”, Feb. 1, 2008, Vol. 47, No. 4 APPLIED OPTICS.

### SUMMARY OF THE INVENTION

In the case where the acoustic wave generated in the internal portion of the object is detected and information on the internal portion of the object is acquired based on the acoustic wave, it is required to maximize a detection sensitivity of the acoustic wave in order to improve an S/N ratio.

On the other hand, in the acoustic wave detector that uses the Fabry-Perot interference film, the wavelength of the measurement light with which the high detection sensitivity can be obtained changes depending on the thickness of the interference film.

That is, in the case where the thickness of the interference film varies or in an environment in which the thickness of the interference film changes due to change of temperature, the optimum wavelength of the measurement light is not determined uniquely.

To cope with this problem, the wavelength of the measurement light is adjusted on an as-needed basis such that the detection sensitivity is optimized in the device described in Non patent Literature 1. Specifically, by changing the wavelength with time and monitoring a change in the light amount of the reflected light of the interference film, measurement is performed while a search for the wavelength that allows the high detection sensitivity of the acoustic wave is performed. However, in the case where this method is used, another problem occurs that time required for the measurement is increased. In particular, in the case where a three-dimensional image representing an optical characteristic value distribution in the internal portion of the object is generated, since it is necessary to acquire a photoacoustic wave signal at a plurality of positions, it is necessary to perform the wavelength search process at a plurality of positions, and the time required for the measurement is increased. In the case where the biological body serves as a measurement target, it is preferable to complete the measurement as quickly as possible such that a strain is not applied to the biological body to be measured.

On the other hand, in the device described in Patent Literature 1, broadband light is split and the optimum wavelength is determined by using light of each wavelength. According to this configuration, it is not necessary to perform the search while changing the wavelength, and hence it is possible to reduce the measurement time.

However, in the configuration, since it is necessary to install a plurality of light detecting sensors at individual measurement positions, when it is intended to improve resolution, the configuration of the device is complicated and cost is increased.

The present invention has been achieved in view of the problem of the conventional art described above, and an object thereof is to provide an acoustic wave detection device capable of obtaining a high detection sensitivity at low cost.

The present invention in its one aspect provides an acoustic wave detection device irradiating measurement light to an interference film and measuring an intensity of an acoustic wave incident on the interference film based on an intensity of the measurement light reflected by the interference film, the acoustic wave detection device comprises the interference film formed of two opposing reflective layers; a first light source configured to irradiate light to at least two different positions of the interference film and capable of changing a wavelength of the light; a photodetector configured to detect an intensity of reflected light which is the light reflected by the interference film; a signal acquisition unit configured to acquire an electrical signal corresponding to the acoustic wave incident on the interference film based on a change of the intensity of the reflected light detected by the photodetector; a wavelength acquisition unit configured to acquire a reference wavelength, which is a wavelength of the measurement light emitted when measurement is performed on a first point on the interference film; and a wavelength control unit configured to determine the wavelength of the measurement light emitted when the measurement is performed on a second point on the interference film based on the reference wavelength, and set the determined wavelength in the first light source.

The present invention in its another aspect provides a photoacoustic measurement device comprises an interference film formed of two opposing reflective layers; a first light source configured to irradiate measurement light to at least two different positions of the interference film and capable of changing a wavelength of light; a photodetector configured to detect an intensity of reflected light which is the measurement light reflected by the interference film; a second light source configured to irradiate excitation light to an object; a signal acquisition unit configured to acquire an electrical signal corresponding to an acoustic wave that is generated from an internal portion of the object due to the excitation light and is incident on the interference film, based on a change of the intensity of the reflected light detected by the photodetector; a wavelength acquisition unit configured to acquire a reference wavelength, which is a wavelength of the measurement light emitted when measurement is performed on a first point on the interference film; a wavelength control unit configured to determine the wavelength of the measurement light emitted when the measurement is performed on a second point on the interference film based on the reference wavelength, and set the determined wavelength in the first light source; and an information generation unit configured to acquire characteristic information on the internal portion of the object based on the electrical signal acquired by the signal acquisition unit.

The present invention in its another aspect provides an acoustic wave detection method performed by an acoustic wave detection device having an interference film formed of two opposing reflective layers, a first light source configured to irradiate light to at least two different positions of the interference film and capable of changing a wavelength of the light, and a photodetector configured to detect an intensity of the light reflected by the interference film, the acoustic wave detection method comprises a wavelength acquisition step of acquiring a reference wavelength, which is a wavelength of measurement light emitted when measurement is performed on a first point on the interference film; a wavelength control step of determining the wavelength of the measurement light emitted when the measurement is performed on a second point on the interference film based on the reference wavelength, and setting the determined wavelength in the first light source; an irradiation step of irradiating the measurement light to the interference film from the first light source; and a signal acquisition step of acquiring an electrical signal corresponding to an acoustic wave incident on the interference film based on a change of the intensity of the reflected light detected by the photodetector.

According to the present invention, it is possible to provide the acoustic wave detection device capable of obtaining the high detection sensitivity at low cost.

Further features of the present invention will become apparent from the following description of exemplary embodiments with reference to the attached drawings.

### BRIEF DESCRIPTION OF THE DRAWINGS

FIG. 1 is a system configuration diagram of a photoacoustic measurement device according to a first embodiment;

FIG. 2 is a view illustrating a detection sensitivity of an acoustic wave;

FIG. 3 is a process flowchart of the photoacoustic measurement device according to the first embodiment;

FIG. 4 is a process flowchart of the photoacoustic measurement device according to the first embodiment;

FIG. 5 is a view showing a relationship between the wavelength of measurement light and a measurement sensitivity;

FIG. 6 is a view illustrating a disposition of a reference point in the first embodiment;

FIG. 7 is a process flowchart of the photoacoustic measurement device according to a second embodiment;

FIG. 8 is a view illustrating the disposition of the reference point in the second embodiment; and

FIG. 9 is a system configuration diagram of an ultrasonic measurement device according to a third embodiment.

### DESCRIPTION OF THE EMBODIMENTS

Hereinbelow, with reference to the drawings, embodiments of the present invention will be described in detail. Note that numeric values, materials, shapes, dispositions and the like used in the description of the embodiments should be appropriately changed depending on configurations and various conditions of devices to which the invention is applied, and the scope of the invention is not limited thereto.

### First Embodiment

A photoacoustic measurement device according to a first embodiment is a device that visualizes, e.g., images optical characteristic information on an internal portion of an object by irradiating pulsed light to the object, and receiving and analyzing a photoacoustic wave generated in the object due to the pulsed light. An example of the optical characteristic information on the internal portion of the object includes an initial sound pressure distribution, a light energy absorption density distribution, or an absorption coefficient distribution derived therefrom.

<System Configuration>

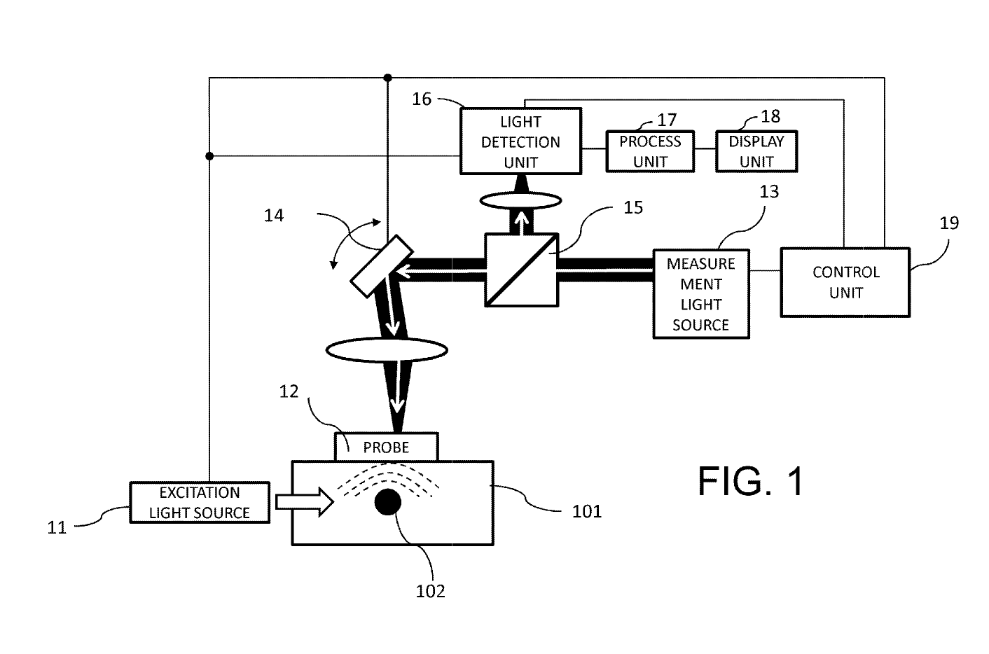

With reference to FIG. 1, the configuration of the photoacoustic measurement device according to the present embodiment will be described. The photoacoustic measurement device according to the present embodiment has an excitation light source 11, a probe 12, a measurement light source 13, a scanning unit 14, a half mirror 15, a light detection unit 16, a process unit 17, a display unit 18, and a control unit 19.

Hereinbelow, a summary of a method for acquiring object information will be described while individual units constituting the photoacoustic measurement device according to the present embodiment will be described.

The excitation light source 11 is a unit that generates pulsed light and irradiates the pulsed light to an object.

The excitation light source 11 is preferably a laser light source in order to obtain a large output, but it is also possible to use a light-emitting diode, a flash lamp and the like instead of the laser. In the case where the laser is used as the light source, it is possible to use various lasers such as a solid state laser, gas laser, dye laser, and semiconductor laser.

Ideally, it is preferable to use an Nd: YAG excitation OPO laser, dye laser, Ti:sa laser, or alexandrite laser in which an output is high and the wavelength can be changed continuously. In addition, a plurality of single-wavelength lasers having different wavelengths may also be used.

The wavelength of the pulsed light is preferably a specific wavelength that allows absorption by a specific component among components constituting the object, and is preferably a wavelength that allows light to propagate to the internal portion of the object. Specifically, in the case where the object is a biological body, the wavelength is preferably not less than 700 nm and not more than 1100 nm. However, it is also possible to use a wavelength range that is wider than the above wavelength range such as, e.g., a wavelength range from 400 nm to 1600 nm, and also use a terahertz wave range, microwave range, and radio wave range.

In order to generate the photoacoustic wave effectively, light needs to be irradiated to the object in sufficiently short time in accordance with thermal characteristics of the object. In the case where the object is the biological body, the pulse width of the pulsed light generated from the light source is preferably about 5 to 50 nanoseconds.

Note that the excitation light source 11 does not necessarily need to be part of the photoacoustic measurement device according to the present embodiment, and may also be connected to the outside.

The pulsed light emitted from the excitation light source 11 is irradiated to an object 101. The object 101 may be part of a biological body (e.g., a breast), or may also be, for example, a phantom obtained by simulating characteristics of the biological body. In the case where the object 101 is the biological body, it is possible to image a light-absorbing body in an internal portion of the biological body or the light-absorbing body on a surface of the biological body such as a tumor and a blood vessel. When the pulsed light is irradiated to the object 101, the light-absorbing body (a reference numeral 102) in the internal portion or on the surface of the object 101 absorbs part of energy of the pulsed light and thereby generates the photoacoustic wave.

The probe 12 is a Fabry-Perot acoustic wave probe. The probe 12 has a optical interference film formed of two opposing reflective layers. Specifically, the probe 12 has a structure in which a polymer film is sandwiched between two resonance mirrors. When the acoustic wave becomes incident on the probe 12, the mirror on the side of the incidence of the acoustic wave deforms in a thickness direction of the polymer film, and a distance between the two mirrors changes. It is possible to detect the sound pressure of the incident acoustic wave by detecting the change of the distance. Specifically, light (measurement light) for measuring the change of the distance between the mirrors is applied to the probe 12 from a surface opposite to a surface on which the acoustic wave is incident, and the change of the intensity of light as the reflected measurement light (hereinafter referred to as reflected light) is detected. With this, it is possible to detect the change of the sound pressure applied to the probe 12.

The measurement light source 13 is a wavelength variable laser device capable of changing the wavelength of emitted laser light, and is a unit that generates laser light having the wavelength determined in the control unit 19 described later. The laser light having a specific wavelength that is generated from the measurement light source 13 is used as the measurement light. A method for determining the wavelength of the measurement light will be described later.

The photoacoustic measurement device according to the present embodiment is capable of setting a position to which the measurement light is irradiated (hereinafter referred to as an irradiation position) at any position on the probe 12.

The scanning unit 14 is a unit that changes the irradiation position of the measurement light on the probe 12. The scanning unit 14 includes an optical member such as a movable mirror (e.g., a galvanometer mirror), and is capable of moving the irradiation position of the measurement light to two or more positions on the probe (on the interference film) by moving the optical member.

The measurement light irradiated to the surface of the probe 12 is reflected by the resonance mirror, and becomes incident on the light detection unit 16 through the half mirror 15.

The light detection unit 16 is a unit that measures the intensity of the incident light (a photodetector and a signal acquisition unit in the present invention). When the acoustic wave becomes incident on the probe 12, the distance between the resonance mirrors changes and the intensity of the reflected light changes. By measuring the intensity of the reflected light, it is possible to convert a sound pressure fluctuation of the acoustic wave incident on the probe 12 to an electrical signal. Hereinbelow, the electrical signal indicative of the sound pressure fluctuation of the acoustic wave is referred to as a photoacoustic signal. Note that the light detection unit 16 may have a unit that amplifies the photoacoustic signal and a unit that converts an analog electrical signal to a digital signal. Note that the photoacoustic signal in the present specification is a concept that includes both of the analog electrical signal obtained in the light detection unit 16 and the converted digital signal.

The photoacoustic signal generated by the light detection unit 16 is a signal indicative of a fluctuation of the amount of the reflected light at one point on the probe 12. The light detection unit 16 transmits corresponding coordinates on the probe to the process unit 17 together with the photoacoustic signal.

In the present embodiment, the above-described operation is performed repeatedly while the irradiation position of the measurement light is changed by using the scanning unit 14. That is, a plurality of photoacoustic signals acquired at a plurality of positions on the probe 12 are transmitted to the process unit 17.

Note that, in the following description, the measurement is used as a word that denotes conversion of the sound pressure of the acoustic wave incident on the probe 12 to the photoacoustic signal unless otherwise stated.

The process unit 17 is a unit (an information generation unit in the present invention) that temporarily stores information transmitted from the light detection unit 16, and generates (reconstructs) an image that represents information on the internal portion of the object, e.g., an optical characteristic value distribution based on the stored information. Examples of the method of the reconstruction include Fourier transformation, universal back projection (UBP), filtered back projection, and phasing addition, and any method may be used. Note that, in the case where there is an area where the film thickness is extremely abnormal (e.g., the case where a foreign substance is present in an element), the process may be performed after data obtained in the area is excluded when the image is reconstructed.

The display unit 18 is a device that displays information generated by the process unit 17 in the form of the image, and a liquid crystal display is typically used as the display unit 18, but displays having other systems such as a plasma display, organic EL display, and FED may also be used.

The control unit 19 is a unit that controls the above-described individual units (a wavelength control unit and a wavelength acquisition unit in the present invention). Specifically, the control unit 19 controls the irradiation intensity, irradiation timing, and irradiation position of each of the excitation light irradiated to the object and the measurement light irradiated to the probe. In addition, the control unit 19 also controls the reception timing of the acoustic wave. The control unit 19 may be implemented by hardware designed for exclusive use or may also be implemented by a software module. Further, the control unit 19 may also be implemented by a field programmable gate array (FPGA) or an application specific integrated circuit (ASIC), or may also be implemented by a combination thereof.

Note that the irradiation position of the measurement light is changed by using the scanning unit 14 in the photoacoustic measurement device according to the present embodiment, but other configurations may be used as long as the configuration guides the measurement light to any position on the probe 12. For example, a configuration in which the irradiation position of the measurement light is changed by using a light emitting element array capable of emitting light beams having a plurality of wavelengths as the measurement light source and switching between the light emitting elements may be used.

<Description of Optimum Wavelength>

Before describing a specific measurement method, the relationship between the detection sensitivity of the acoustic wave and the wavelength in the Fabry-Perot probe will be described.

The Fabry-Perot probe acquires the fluctuation of the distance between interference mirrors that occurs due to the sound pressure of the incident acoustic wave by detecting the intensity of the reflected light of the measurement light.

Herein, a reflectance (Iᵣ/Iᵢ) in the case where it is assumed that the intensity of the measurement light irradiated to the probe 12 is Iᵢ, and the intensity of the reflected light is Iᵣ can be represented by Expression (1).

$$\begin{matrix}[ Math . 1 ] & \\ \frac{{I}_{r}}{{I}_{i}} = \frac{4 R {sin}^{2} \frac{ϕ}{2}}{{( 1 - R )}^{2} + 4 R {sin}^{2} \frac{ϕ}{2}} & Expression ( 1 )\end{matrix}$$

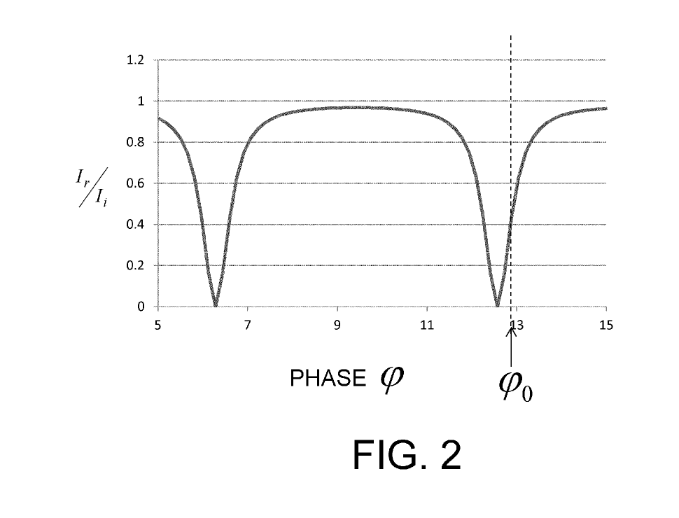

R in Expression (1) is the reflectance of the mirror of the probe 12, and φ is a phase. Herein, when it is assumed that the wavelength of the measurement light is λ, the refractive index of a spacer film stacked between the interference mirrors is n, and the distance between the mirrors is d, the relationship of Expression (2) is established. FIG. 2 is a graph representing this relationship.

φ=(4π/λ)×nd Expression (2)

In the graph in FIG. 2, the horizontal axis indicates the phase, and the vertical axis indicates the reflectance. As can be seen from the graph, the phase that maximizes the change of the reflectance to the change of the phase is φ₀ in the drawing. Incidentally, as can be seen from Expression (2), the relationship between the phase and the distance between the mirrors d is linear. Consequently, the change of the reflectance to a minute fluctuation of the distance between the mirrors d caused by the sound pressure of the acoustic wave is maximized when the phase is φ₀. That is, the detection sensitivity of the acoustic wave is maximized when the phase φ₀ is realized. That is, in the case where a given distance between the mirrors d is assumed, the wavelength (hereinafter referred to as an optimum wavelength) λ₀ that maximizes the detection sensitivity can be represented by Expression (3).

λ₀=(4π/φ₀)×nd Expression (3)

However, the optimum wavelength is not necessarily the same at all of the positions on the probe.

One of the reasons for that is a fluctuation of temperature. Since the interference film is deformed by heat depending on the temperature, the film thickness changes with the deformation, and the optimum wavelength that depends on the film thickness changes. For example, when the biological body as the object comes into contact with the probe, the temperature of the probe fluctuates due to body temperature, and the optimum wavelength fluctuates due to the fluctuation. In addition, the fluctuation of the optimum wavelength is also caused by change of an ambient temperature.

Another reason therefore is variations in the thickness of the interference film of the probe. The variations in the film thickness occur mainly during manufacturing. In the case where the film thickness is not uniform, the optimum wavelength differs from one measurement position to another.

Thus, the optimum wavelength may differ from one measurement position to another, and the optimum wavelength may change with time even at the same measurement position. To cope with this, it is necessary to search for the optimum wavelength at each measurement position and cause the wavelength of the measurement light to match the optimum wavelength in order to realize the measurement having a high sensitivity.

<Summary of Measurement>

The photoacoustic measurement device according to the present embodiment has information on a plurality of measurement positions (hereinafter referred to as measurement points) set on the probe 12, and acquires the distribution of the sound pressure of the acoustic wave incident on the probe by irradiating the measurement light to the measurement points sequentially.

In addition, some of the plurality of measurement points are set as reference points (a first point in the present invention) and, when the photoacoustic measurement device measures the reference point, the photoacoustic measurement device performs a search for the optimum wavelength. In addition, at the other measurement points (a second point in the present invention. Hereinafter referred to as a non-reference point), the photoacoustic measurement device performs the measurement by using the optimum wavelength acquired at the reference point (hereinafter referred to as a reference wavelength) without performing the search for the optimum wavelength.

Information on positions of the measurement point and the reference point is pre-stored in the control unit 19 together with the order of the measurement. Note that a specific example of the disposition of the measurement point and the reference point will be described later.

<Process Flowchart>

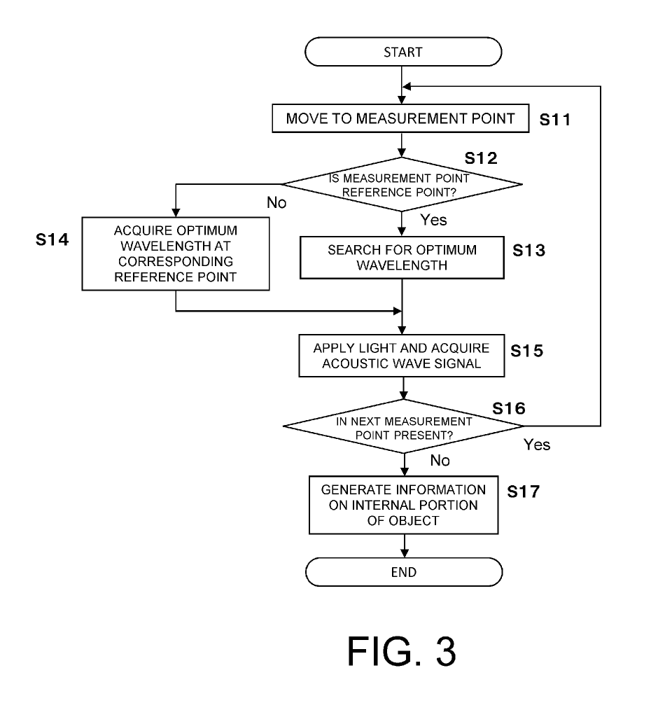

A description will be given of the method of the measurement performed on the object by the photoacoustic measurement device according to the present embodiment with reference to FIG. 3 as a process flowchart.

First, in Step S11, the measurement point to which the measurement light is irradiated is determined, and the irradiation position of the measurement light is moved to the measurement point by using the scanning unit 14.

Next, in Step S12, it is determined whether or not the target measurement point is the point set as the reference point. In the case where the target measurement point is the reference point, the process of searching for the optimum wavelength is executed in Step S13, and the optimum wavelength at the reference point is determined. The process executed in Step S13 is the process in which wavelength sweeping is performed on the entire range where the optimum wavelength may be present, and the search for the optimum wavelength is performed. Hereinafter, such a process is called entire range sweeping.

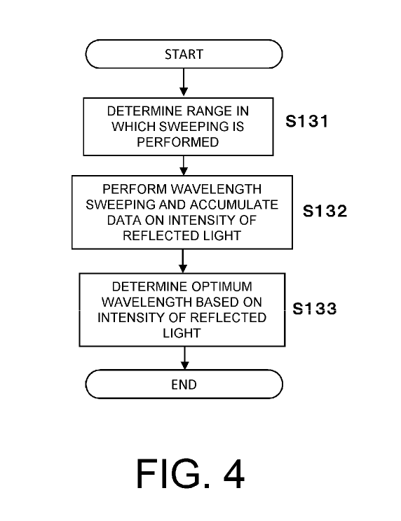

FIG. 4 is a flowchart specifically showing the search process of the optimum wavelength performed in Step S13 in detail. In Step S13, light for performing auxiliary measurement (hereinafter referred to as auxiliary measurement light) is irradiated to any measurement position on the probe, the change of the amount of the reflected light is recorded while the wavelength of the light is changed, and the optimum wavelength is calculated based on the result.

First, in Step S131, a wavelength sweeping range for searching for the optimum wavelength is set. Specifically, a predicted value of the optimum wavelength calculated from design values of the interference film is acquired first. Next, the maximum value of the variations in the thickness of the interference film and a fluctuation predicted amount of the wavelength calculated from the maximum temperature difference are acquired. Then, the fluctuation predicted amount is applied to the predicted value of the optimum wavelength, and the wavelength sweeping range is determined.

Next, in Step S132, the wavelength sweeping is performed. The wavelength sweeping in the present specification denotes an operation in which the auxiliary measurement light is irradiated to the probe from the measurement light source 13 a plurality of times while the wavelength is changed in the set wavelength sweeping range, and the intensity of the reflected light is acquired for each wavelength.

Lastly, in Step S133, the optimum wavelength is calculated by the above-described method based on the intensity of the reflected light obtained as the result of the wavelength sweeping. For example, it is possible to differentiate acquired data (data indicative of a wavelength dependence of the amount of the reflected light) with the wavelength and determine the wavelength having the maximum value as the optimum wavelength.

Note that, as the method for calculating the optimum wavelength, methods other than the method described byway of example may also be used as long as the wavelength that allows the maximum sensitivity can be identified. The optimum wavelength determined in Step S133 is temporarily stored in association with the position of the reference point.

Returning to FIG. 3, the description will be continued.

In the case where the target measurement point is not the reference point in the determination in Step S12, the process is shifted to Step S14, and the optimum wavelength at the corresponding reference point (the reference wavelength) is acquired. The reference wavelength acquired in each of Steps S13 and S14 is set in the measurement light source 13.

In Step S15, the measurement light having the set wavelength is irradiated to the measurement point on the probe 12, and the pulsed light generated in the excitation light source 11 is irradiated to the object. The irradiated measurement light is reflected by the probe 12, and the intensity (the light amount) of the reflected light is detected by the light detection unit 16. Note that the detection of the light amount performed by the light detection unit 16 is performed in synchronization with a light emission trigger signal outputted from the excitation light source 11. The change of the amount of the reflected light (the photoacoustic signal) detected by the light detection unit 16 is accumulated in a memory in the process unit 17.

In Step S16, it is determined whether or not it is necessary to perform the measurement at other measurement points and, in the case where the measurement point that is not subjected to the process is present, the process is shifted to Step S13, and the measurement is continued. In the case where all of the measurement is completed, the process is shifted to Step S17.

In Step S17, the process unit 17 performs image reconstruction by using the accumulated photoacoustic signal, and generates a three-dimensional image representing a light energy absorption density distribution in the internal portion of the object. Further, after the process unit 17 performs proper image processing, the process unit 17 outputs the image to the display unit 18.

<Disposition Method of Reference Point>

As described above, the photoacoustic measurement device according to the present embodiment divides a plurality of measurement points positioned on the probe into the measurement point (the reference point) at which the search for the optimum wavelength is performed and the measurement point (the non-reference point) at which the search for the optimum wavelength is not performed, and reduces the measurement time by reusing the optimum wavelength obtained by the search. However, in the case where the target measurement point is too far away from the reference point, the deviation of the optimum wavelength may exceed a tolerance. To cope with this, the photoacoustic measurement device according to the present embodiment sets the reference point such that the deviation amount of the optimum wavelength falls within a predetermined range.

A description will be given by using a specific example.

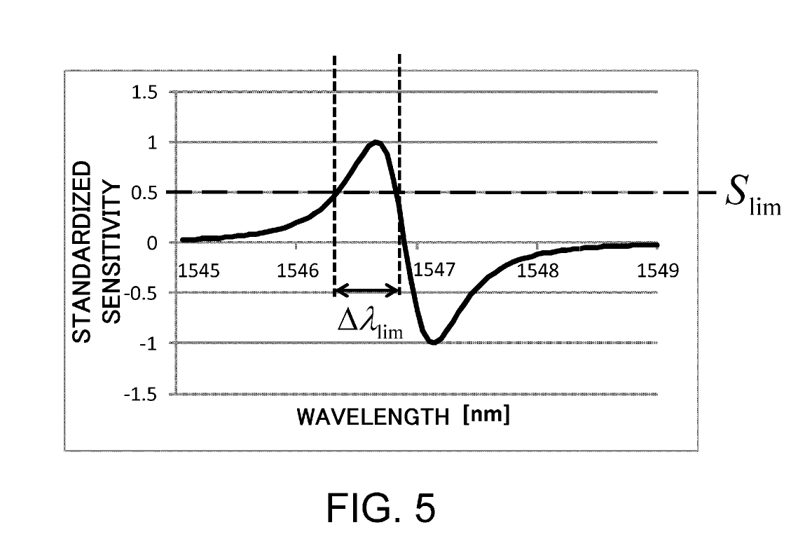

FIG. 5 is a view showing a relationship between the wavelength of the measurement light (horizontal axis) and a normalized measurement sensitivity (vertical axis). Note that the measurement sensitivity is proportional to a value obtained by differentiating the reflectance with the phase φ. FIG. 5 shows characteristics in the vicinity of the wavelength=1550 [nm] in the case where the reflectance of the interference film is 0.9, the refractive index of the interference film is 1.65 (parylene film), and the film thickness is 30 [um]. Note that parameters other than those mentioned above may be used.

As shown in FIG. 5, when it is assumed that the permissible minimum sensitivity that is represented by a ratio to the maximum sensitivity (hereinafter referred to as a permissible sensitivity) is Sₗᵢₘ, the width of the wavelength that allows the sensitivity of not less than Sₗᵢₘ is Δλₗᵢₘ.

In addition, it is assumed that the maximum value of a difference between any wavelength included in the wavelength width and the wavelength that allows the maximum sensitivity (the optimum wavelength) is Δλₘₐₓ, and the minimum value thereof is Δλₘᵢₙ.

The reference point may be appropriately set within a range in which the change amount of the optimum wavelength resulting from the movement on the interference film is not more than Δλₗᵢₘ.

Main factors for the deviation of the optimum wavelength include the change of the temperature, the change of the thickness of the interference film caused by the temperature change, and variations in film thickness during the manufacturing of the interference film, as described above.

Herein, when it is assumed that the change amount of the film thickness to a movement amount Δl on the interference film is (Δd/Δl), the change amount can be represented by Expression (4).

$$\begin{matrix}[ Math . 2 ] & \\ \frac{Δ d}{Δ l} = kd ( \frac{Δ {T}_{x}}{Δ l} + \frac{Δ {T}_{t}}{Δ l} ) + \frac{Δ {d}_{x}}{Δ l} & Expression ( 4 )\end{matrix}$$

Herein, k is a coefficient of thermal expansion [1/° C.] of the fabry-Perot interference film, and ΔTₓ/Δl is the change amount [° C./m] of the temperature to the movement amount on the interference film that results from the initial temperature distribution. In addition, ΔTₜ/Δl is the change amount [° C./m] of the temperature to the movement amount on the interference film that results from a lapse of time. Note that ΔTₜ/Δl is the amount that depends on time of measurement of the acoustic wave, and hence ΔTₜ/Δl depends on the scanning speed and the scanning method of the measurement light.

Further, Δdₓ/Δl is the change amount [m/m] (resulting from variations in film thickness during manufacturing) of the film thickness to the movement amount on the interference film.

From Expression described above, the difference Δλ in the optimum wavelength between two points having a film thickness difference of Δd is represented by Expression (5).

Δλ=(4π/φ)₀)×nΔd Expression (5)

Consequently, the movement amount that allows the sensitivity of not less than the permissible sensitivity Sₗᵢₘ is a movement amount lₗ that satisfies Δλₘᵢₙ≦Δλ≦Δλₘₐₓ. When lₘᵢₙ and lₘₐₓ are represented by Expression (6) and Expression (7), lₘᵢₙ≦l_≦lₘₐₓ is established.

$$\begin{matrix}[ Math . 4 ] & \\ {l}_{min} = \frac{Δ {λ}_{min} {ϕ}_{0}}{4 π n ( \frac{Δ d}{Δ l} )} & Expression ( 6 ) \\ [ Math . 5 ] & \\ {l}_{max} = \frac{Δ {λ}_{max} {ϕ}_{0}}{4 π n ( \frac{Δ d}{Δ l} )} & Expression ( 7 )\end{matrix}$$

When the foregoing is marshaled, it can be seen that, in the case where the distance from the target measurement point to the reference point is assumed to be lₗ, it is possible to assure the permissible sensitivity Sₗᵢₘ by disposing the reference point in the range that satisfies lₘᵢₙ≦lₗ≦lₘₐₓ.

Note that only the one-dimensional direction on the interference film has been described in the above example, and the same description can be applied to two-dimensional coordinates, and hence the description thereof will be omitted.

Note that the range of lₘᵢₙ≦lₗ≦lₘₐₓ is assumed to be sufficiently small in the above example, and hence it is assumed that Δd/Δl is constant in the range. However, even in the case where this assumption is not satisfied, the same description as that in the above example can be used, and hence the description in that case will be omitted.

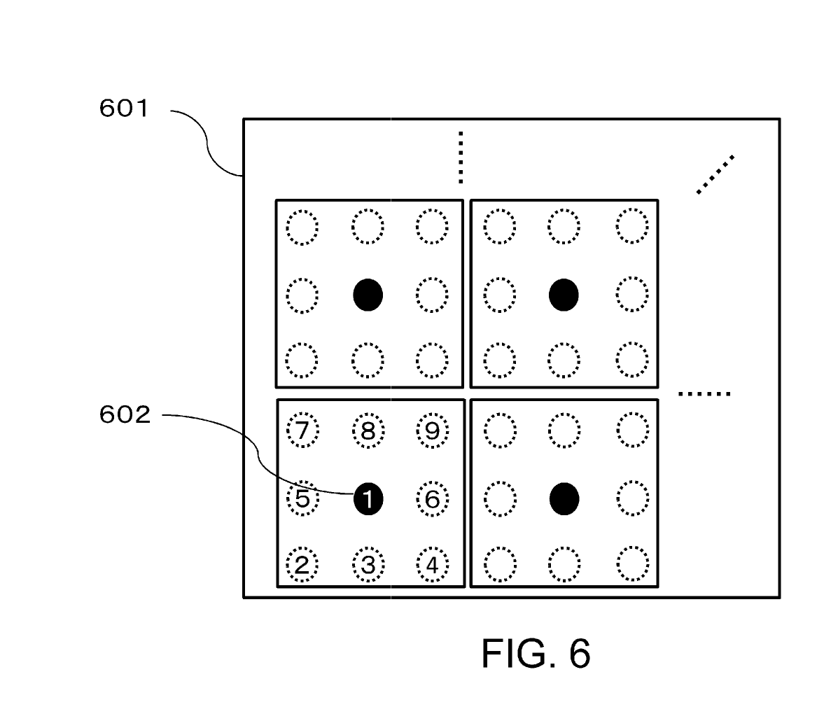

FIG. 6 shows an example in which a plurality of measurement points and a plurality of reference points are disposed on the probe 12 such that the above-described condition is satisfied. A reference numeral 601 indicates an area (hereinafter referred to as a measurement area) on the probe 12 to which the measurement light is irradiated. The measurement point indicated by a reference numeral 602 is a point at which the measurement is started. Numbers given to the measurement points denote the order of the measurement.

Note that the point indicated by a solid circle is the reference point and the point indicated by a dotted line is the non-reference point. A square area including nine measurement points conrresponds to an area where the measurement is performed by using the same wavelength. That is, by using the optimum wavelength at the reference point positioned at the center of the square area, the measurement of the other eight measurement points is performed. When the measurement of the nine measurement points is ended, the measurement position moves to the next square area, and the same process is repeated.

Note that the disposition method of the reference point is not limited to the method described by way of example as long as the above-described relationship of lₘᵢₙ≦lₗ≦lₘₐₓ is satisfied. In addition, the order of the measurement is not limited to the order thereof described by way of example. However, it is preferable to make considerations such that the temperature change of the probe does not become abrupt.

In addition, instead of using the arithmetic calculation described above, the position of the reference point may also be determined by actually measuring the change of the measurement sensitivity to the distance on the interference film.

Next, a description will be given of effects obtained in the first embodiment.

According to the result of a study conducted by the inventors, the permissible sensitivity Sₗᵢₘ is sufficient if it is not less than 0.5. Δλₗᵢₘ in this case is about 0.5 [nm].

It is assumed that the change amount of the film thickness to the movement amount on the interference film is 10⁻⁶ [m/m], and the temperature change amount on the interference film is 50[° C./m]. Note that an effect of the temporal change of the probe temperature exerted on the optimum wavelength during scanning between the reference points is not considered.

In addition, parylene is used in the interference film of the probe. In the case of the conditions described above, the range of lₗ is about±250 [um] at the maximum.

When it is assumed that the speed of the wavelength sweeping is 100 [nm/s], a sweeping width is 10 [nm], and an irradiation interval of the measurement light is 100 [um], time required for at least one wavelength sweeping is 100 [ms]. Consequently, in the case where the measurement is performed on a two-dimensional plane of 100×100 step, when the wavelength sweeping is performed at all of the measurement points, time of about 1000 [s] is required.

On the other hand, in the case where the measurement is performed according to the pattern shown in FIG. 6, the process amount is 1/9 of the process amount in the case where the wavelength sweeping is performed at all of the measurement points, and hence it is possible to reduce the process time to about 111 [s]. As a matter of course, it is possible to reduce the measurement time in environments other than the measurement environment assumed in the foregoing.

Thus, in the first embodiment, a pluraliy of the measurement points positioned on the probe are divided into the measurement point (the reference point) at which the search for the optimum wavelength is performed and the measurement point (the non-reference point) at which the search for the optimum wavelength is not performed, and the optimum wavelength obtained by the search is reused in the measurement of the non-reference point. With this configuration, it is possible to prevent a reduction in detection sensitivity resulting from variations in the temperature of the interference film and variations in film thickness, and reduce the measurement time.

### Second Embodiment

In the first embodiment, the measurement of the non-reference point has been performed by using the reference wavelength acquired at the reference point. However, since the reference point and the non-reference point are spaced apart from each other, a significant fluctuation of the film thickness or the temperature may occur and the deviation from the truly optimum wavelength may occur.

To cope with this problem, a second embodiment is an embodiment that sweeps the wavelengths before and after the reference wavelength and determines the truly optimum wavelength instead of using the reference wavelength acquired at the reference point without alteration. The sweeping performed in the second embodiment is not the entire range sweeping described by way of example in the first embodiment, but is the sweeping for correcting a slight deviation of the wavelength in which the range is limited.

The system configuration diagram of the photoacoustic measurement device in the second embodiment is the same as that of the first embodiment, and hence the detailed description thereof will be omitted, and only points of the process different from those in the first embodiment will be described.

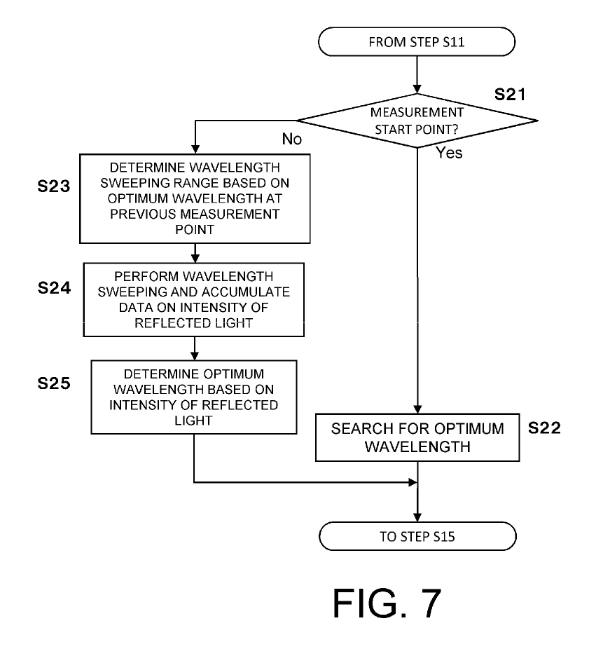

In the second embodiment, the processes in Steps S12, S13, and S14 described in the first embodiment are replaced with those shown in FIG. 7.

In Step S21, it is determined whether or not the measurement point is the point at which the measurement is started (a measurement start point). In the case where the measurement point is the measurement start point, the process is shifted to Step S22, and the search for the optimum wavelength is performed by the same process as that in Step S13. In the case where the measurement point is not the measurement start point, the process is shifted to Step S23, the optimum wavelength (the reference wavelength) at the previous measurement point is acquired, and the wavelength sweeping range is determined based on the reference wavelength. Hereinbelow, a specific method will be described.

Herein, when it is assumed that the change amount of the film thickness to the movement amount Δl on the interference film is (Δd/Δl), and the distance between the reference point and the non-reference point is l₂, the absolute value Δλ_{range} of the change amount of the optimum wavelength is represented by Expression (8).

$$\begin{matrix}[ Math . 6 ] & \\ Δ {λ}_{range} = \frac{4 π}{{ϕ}_{0}} n \vert Δ d \vert = \frac{4 π n}{{ϕ}_{0}} \vert \frac{Δ d}{Δ l} \vert {l}_{2} & Expression ( 8 )\end{matrix}$$

When the reference wavelength is assumed to be λ₀, the wavelength sweeping range required to determine the optimum wavelength is a range represented by λ₀−Δλ_{range}≦λ≦λ₀+Δλ_{range}.

Processes in Steps S24 and S25 are the same as those in Steps S132 and S133, and hence the description thereof will be omitted.

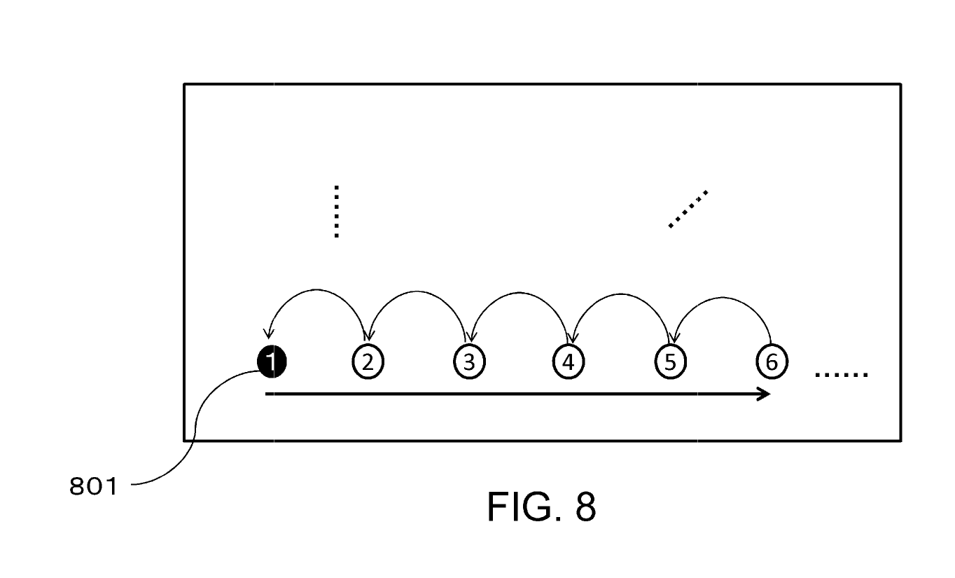

FIG. 8 is a view showing disposition positions of a plurality of measurement points and reference points on the probe 12. The measurement point indicated by a reference numeral 801 is the point at which the measurement is started (the measurement start point), and numbers given to the measurement points denote the order of the measurement. In the present embodiment, the non-reference point at which the measurement is performed at a given timing serves as the reference point when the next measurement point is measured.

Thus, in the second embodiment, the reference wavelength is acquired by using the previous measurement point as the reference point, the range of the wavelength is narrowed down based on the reference wavelength, and the wavelength sweeping is performed. With this, it is possible to acquire the optimum wavelength at each measurement point accurately at high speed.

Note that the wavelength sweeping range is determined while the previous measurement point is referred to in the present embodiment, but the reference point does not necessarily need to be the previous measurement point. For example, it is also possible to use the second previous measurement point or the distant measurement point as the reference point. Alternatively, the other measurement point at which the measurement has been performed may be used as the reference point.

In addition, the value of Δλ_{range} may be calculated from known parameters, or may also be determined by actual measurement. The entire range sweeping has been performed only at the measurement start point in the second embodiment, but the entire range sweeping may also be performed at any measurement point other than the point at the position described by way example. For example, in the case where a plurality of measurement points are disposed in a plurality of rows, the entire range sweeping may be performed every time the measurement position moves to the next row. In addition, the entire range sweeping may be performed every time the measurement is performed a predetermined number of times.

In the case where the optimum wavelength cannot be determined in the wavelength sweeping range set in Step S23, the search may also be performed again after the wavelength sweeping range is expanded. In this case, the upper limit of the range may be raised and the lower limit thereof may be lowered with the value of Δλ_{range} used as the reference.

Next, a description will be given of effects obtained in the second embodiment. Herein, the case where the measurement is performed by the method shown in FIG. 8 with the same parameters as those in the first embodiment will be studied.

When it is assumed that the speed of the wavelength sweeping is 100 [nm/s], Δλ_{range} is 0.1 [nm], and the irradiation interval of the measurement light is 100 [um], since the range of ±0.1 [nm] with λ₀ used as the center wavelength is swept, time required for at least one wavelength search is 2 [ms]. Consequently, in the case where the measurement is performed on the two-dimensional plane of 100×100 step, the total time required for the wavelength sweeping is about 20 [s] and, similarly to the first embodiment, it is possible to significantly reduce the measurement time as compared with the conventional art.

Note that, actually, since it takes some time to switch the wavelength of the laser, the total measurement time does not necessarily have the above-described value. However, as compared with the photoacoustic measurement device according to the conventional art, it is possible to significantly reduce the measurement time.

### Third Embodiment

In each of the first and second embodiments, the photoacoustic measurement device that generates the acoustic wave by irradiating the pulsed light to the object has been described. On the other hand, the present invention can also be applied to a device (an acoustic measurement device) that transmits the acoustic wave to the object and acquires information on the internal portion of the object by detecting the reflected acoustic wave. A third embodiment is an embodiment in which the present invention is applied to the acoustic measurement device.

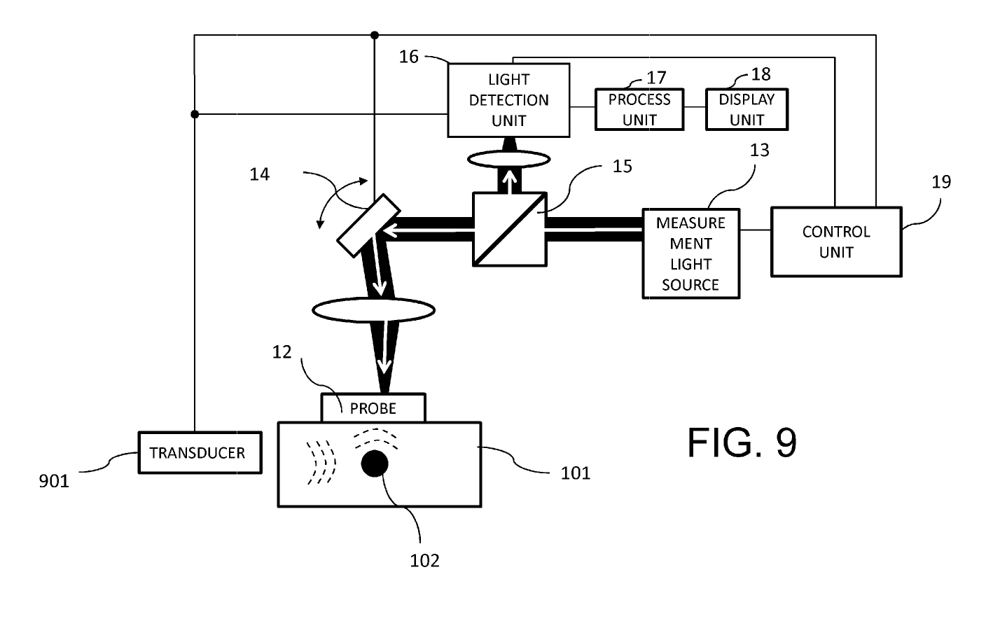

FIG. 9 is a system configuration diagram of an ultrasonic measurement device according to the third embodiment. The ultrasonic measurement device according to the present embodiment is a device that transmits an ultrasonic wave to the object and images an interface having a difference in acoustic impedence in the internal portion of the object by detecting the reflected ultrasonic wave. The same units as those in the first and second embodiments are designated by the same reference numerals, and the description thereof will be omitted.

The ultrasonic measurement device according to the present embodiment has a transducer 901 that transmits the ultrasonic wave to the object. In addition, in the present embodiment, the control unit 19 has the function of controlling the waveform of the ultrasonic wave transmitted from the transducer 901.

In the present embodiment, the probe 12 is a unit that receives the ultrasonic wave reflected in the internal portion of the object. That is, it is possible to detect the sound pressure of the ultrasonic wave reflected at an interface of a tissue having the acoustic impedence different from that of the surrounding area such as a tumor.

Note that a process of generating the image based on the detected ultrasonic wave is the same as that in the first embodiment, and hence the description thereof will be omitted.

According to the third embodiment, in the device (the acoustic measurement device) that images the interface having the difference in acoustic impedence in the internal portion of the object, similarly to the first and second embodiments, it is possible to achieve a reduction in measurement time and an improvement in detection sensitivity.

(Modification)

The descriptions of the embodiments are exemplary descriptions given to illustrate the present invention, and the present invention can be implemented by appropriatly changing or combining the embodiments without departing from the gist of the invention.

For example, the present invention can also be implemented as the photoacoustic measurement device or the acoustic measurement device that includes at least part of the above processes. In addition, the present invention can also be implemented as a control method of the photoacoustic measurement device or the acoustic measurement device that includes at least part of the above processes. Further, the present invention can be implemented as a single acoustic wave detection device or can also be implemented as an acoustic wave detection method executed by the acoustic wave detection device. The above processes and units can be combined arbitrarily as long as no technical conflicts occur.

In each of the first and second embodiments, the excitation light is irradiated to the object from the direction in which the excitation light is not hindered by the probe 12, but the excitation light may also be irradiated to the object via the probe by using the excitation light having a wavelength that allows the excitation light to pass through the mirror of the probe 12.

In addition, an acoustic coupling medium other than that described by way of example may be disposed between the object and the probe. For example, the measurement may be performed by disposing the object in a water tank, and bringing the probe 12 into intimate contact with the surface of water.

Further, in each of the first and second embodiments, the example in which the pulsed light having the single wavelength is irradiated to the object as the excitation light has been described, but pulsed light beams having a plurality of wavelengths may be irradiated to the object and, based on obtained optical coefficients of the internal portion of the object, a concentration distribution of a substance constituting the tissue of the object may be acquired. For example, it is possible to calculate the distribution of a substance concentration in the internal portion of the object, particularly the concentration distributions of oxygen included in blood in a blood vessel, fat, collagen, glucose, and hemoglobin by using photoacoustic signals obtained using a plurality of wavelengths and spectrum information.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment (s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2014-215449, filed on Oct. 22, 2014, which is hereby incorporated by reference herein in its entirety.

What is claimed:

1. An acoustic wave detection device irradiating measurement light to an interference film and measuring an intensity of an acoustic wave incident on the interference film based on an intensity of the measurement light reflected by the interference film, the acoustic wave detection device comprising:
the interference film formed of two opposing reflective layers;
a first light source configured to irradiate light to at least two different positions of the interference film and capable of changing a wavelength of the light;
a photodetector configured to detect an intensity of reflected light which is the light reflected by the interference film;
a signal acquisition unit configured to acquire an electrical signal corresponding to the acoustic wave incident on the interference film based on a change of the intensity of the reflected light detected by the photodetector;
a wavelength acquisition unit configured to acquire a reference wavelength, which is a wavelength of the measurement light emitted when measurement is performed on a first point on the interference film; and
a wavelength control unit configured to determine the wavelength of the measurement light emitted when the measurement is performed on a second point on the interference film based on the reference wavelength, and set the determined wavelength in the first light source.

2. The acoustic wave detection device according to claim 1, further comprising:
a second wavelength control unit configured to, when the measurement is performed on the first point, irradiate the light emitted from the first light source, to the first point a plurality of times as auxiliary measurement light while changing the wavelength, and determine the wavelength of the measurement light irradiated to the first point based on the intensity of the reflected light acquired for each wavelength.

3. The acoustic wave detection device according to claim 2, wherein
when the measurement is performed on the second point, the wavelength control unit determines a sweeping range that is a range in which the wavelength is swept based on the reference wavelength, irradiates the light emitted from the first light source, to the second point a plurality of times as auxiliary measurement light while changing the wavelength in the sweeping range, and determines the wavelength of the measurement light irradiated to the second point based on the intensity of the reflected light acquired for each wavelength.

4. The acoustic wave detection device according to claim 1, wherein
the first point is a point such that the difference in thickness of the interference film between the first point and the second point is smaller than a predetermined value.

5. The acoustic wave detection device according to claim 1, wherein
the first point is a point such that the difference in temperature on the interference film between the first point and the second point is smaller than a predetermined value.

6. A photoacoustic measurement device comprising:
an interference film formed of two opposing reflective layers;
a first light source configured to irradiate measurement light to at least two different positions of the interference film and capable of changing a wavelength of light;
a photodetector configured to detect an intensity of reflected light which is the measurement light reflected by the interference film;
a second light source configured to irradiate excitation light to an object;
a signal acquisition unit configured to acquire an electrical signal corresponding to an acoustic wave that is generated from an internal portion of the object due to the excitation light and is incident on the interference film, based on a change of the intensity of the reflected light detected by the photodetector;
a wavelength acquisition unit configured to acquire a reference wavelength, which is a wavelength of the measurement light emitted when measurement is performed on a first point on the interference film;
a wavelength control unit configured to determine the wavelength of the measurement light emitted when the measurement is performed on a second point on the interference film based on the reference wavelength, and set the determined wavelength in the first light source; and
an information generation unit configured to acquire characteristic information on the internal portion of the object based on the electrical signal acquired by the signal acquisition unit.

7. An acoustic wave detection method performed by an acoustic wave detection device having an interference film formed of two opposing reflective layers, a first light source configured to irradiate light to at least two different positions of the interference film and capable of changing a wavelength of the light, and a photodetector configured to detect an intensity of the light reflected by the interference film, the acoustic wave detection method comprising:
a wavelength acquisition step of acquiring a reference wavelength, which is a wavelength of measurement light emitted when measurement is performed on a first point on the interference film;
a wavelength control step of determining the wavelength of the measurement light emitted when the measurement is performed on a second point on the interference film based on the reference wavelength, and setting the determined wavelength in the first light source;
an irradiation step of irradiating the measurement light to the interference film from the first light source; and
a signal acquisition step of acquiring an electrical signal corresponding to an acoustic wave incident on the interference film based on a change of the intensity of the reflected light detected by the photodetector.

8. The acoustic wave detection method according to claim 7, further comprising a second wavelength control step of, when the measurement is performed on the first point, irradiating the light emitted from the first light source, to the first point a plurality of times as auxiliary measurement light while changing the wavelength, and determining the wavelength of the measurement light irradiated to the first point based on the intensity of the reflected light acquired for each wavelength.

9. The acoustic wave detection method according to claim 7, wherein
when the measurement is performed on the second point in the wavelength control step, a sweeping range that is a range in which the wavelength is swept is determined based on the reference wavelength, the light emitted from the first light source is irradiated to the second point a plurality of times as auxiliary measurement light while the wavelength is changed in the sweeping range, and the wavelength of the measurement light irradiated to the second point is determined based on the intensity of the reflected light acquired for each wavelength.
